(19) United States
(12) Patent Application Publication
(10) Pub. No.: US 2026/0294400 A1
(43) Pub. Date: Oct. 1, 2026

(54) ULTRASOUND DIAGNOSTIC APPARATUS

(71) Applicant: FUJIFILM Corporation, Tokyo (JP)

(72) Inventor: Tatsuya TAKAHASHI, Tokyo (JP)

(73) Assignee: FUJIFILM Corporation, Tokyo (JP)

(21) Appl. No.: 19/570,163

(22) Filed: Mar. 18, 2026

(30) Foreign Application Priority Data
Mar. 31, 2025 (JP) 2025-057453

(51) Int. Cl. A61B 8/00

(52) U.S. Cl. CPC A61B 8/467; A61B 8/465

(57) ABSTRACT

A user sets a movement speed of a pointer for each of a plurality of display regions constituting a subject information input screen related to an ultrasound diagnosis. In the ultrasound diagnostic apparatus, the pointer on the screen is moved by operating a trackball. In this case, in a case in which the pointer is positioned in the display region in which a normal movement speed is set, the pointer is moved at a normal speed. In a case in which the pointer is positioned in the display region in which a movement speed slower than normal is set, the pointer is moved at a slow speed. In a case in which the pointer is positioned in the display region in which a movement speed faster than normal is set, the pointer is moved at a fast speed.

### CROSS-REFERENCE TO RELATED APPLICATIONS

This application is based on and claims priority under 35 USC 119 from Japanese Patent Application No. 2025-057453 filed March 31, 2025.

### 1. FIELD OF THE INVENTION

The present disclosure relates to an ultrasound diagnostic apparatus, particularly, to operation of a pointing device.

### 2. DESCRIPTION OF THE RELATED ART

The ultrasound diagnostic apparatus acquires biological information of a subject by transmitting an ultrasound wave into the subject using an ultrasound probe and receiving a reflected wave thereof. The acquired biological information is displayed as an ultrasound image representing a state of the subject.

In order to manage information such as the generated ultrasound image and a history of the examination, it is necessary to input information related to the subject via the monitor and store the information in a database. The information related to the subject includes personal information such as a name and a date of birth.

A trackball is usually provided as a pointing device on a console panel of the ultrasound diagnostic apparatus used for inputting the information related to the subject. The user can move a pointer displayed on the monitor in an operation direction by operating the trackball to rotate the trackball in a desired direction. The user can suppress a movement amount of the pointer by rotating the trackball slowly, that is, by suppressing a rotation amount. On the other hand, the user can move the pointer to a position farther from a current position by rotating the trackball more.

For example, in a case in which the user inputs the information related to the subject from an input screen of the subject information, the user operates the trackball to move the pointer to a desired input field and inputs predetermined item information from the input field at a position indicated by the pointer.

### SUMMARY OF THE INVENTION

In a case in which the user moves the pointer to a desired position on the screen, the user adjusts an operation amount of the pointing device. A movement speed of the pointer is constant regardless of a position of the pointer on the screen. The movement speed of the pointer may be adjustable. However, the pointer moves on the screen at the adjusted constant movement speed regardless of a position of the pointer on the screen.

Meanwhile, the user may want to finely adjust the position of the pointer on the screen. In this case, the user needs to operate the pointing device by a small amount, but the pointer passes the desired position unless the small amount of adjustment is performed well. On the other hand, the user may want to move the pointer at an end of the screen to the other end at once. In this case, in a case in which the pointing device is the trackball, the user rotates the trackball quickly to move the pointer quickly, but the pointer may not reach the desired position.

An object of the present disclosure is to control a movement speed of a pointer according to a position of the pointer on a display screen of an ultrasound diagnostic apparatus.

An ultrasound diagnostic apparatus according to the present disclosure comprises a processor, and the processor is configured to control a movement speed of a pointer that moves on a screen on which information related to an ultrasound diagnosis is displayed in accordance with a display position of the pointer, the pointer being moved by operation of a pointing device.

In addition, the movement speed of the pointer may be set for each region constituting the screen.

In addition, the processor may move the pointer in accordance with the movement speed set for each region by a user via a predetermined setting screen.

In addition, the processor may control the movement speed of the pointer based on an attribute of a display element present around a position of the pointer.

In addition, the processor may control the movement speed of the pointer in accordance with a result of comparing a position attribute of the pointer obtained from the attribute of the display element with a predetermined threshold value.

According to the present disclosure, the movement speed of a pointer can be controlled according to the position of the pointer on the display screen of the ultrasound diagnostic apparatus.

### BRIEF DESCRIPTION OF THE DRAWINGS

FIG. 1 is a block diagram showing a schematic configuration of an ultrasound diagnostic apparatus according to Embodiment 1.

FIG. 2 is a diagram showing a display example of a subject information input screen used to describe movement of a pointer before applying characteristic movement speed control in Embodiment 1.

FIG. 3 is a diagram showing a display example of a review screen used to describe control of a movement speed according to a position of the characteristic pointer in Embodiment 1.

FIG. 4 is a diagram showing an example of a data configuration of screen information in Embodiment 1.

FIG. 5 is a diagram showing an example of a data configuration of configuration information in Embodiment 2.

FIG. 6 is a diagram showing an example of a layout configuration of a pointer movement speed setting screen in the embodiment.

FIG. 7 is a flowchart showing pointer movement control processing in Embodiment 1.

FIG. 8 is a diagram showing a display example of a subject information input screen used to describe movement of a pointer in a case in which the characteristic movement speed control is applied in Embodiment 1.

FIG. 9 is a diagram showing an example of a data configuration of display element information in Embodiment 2.

FIG. 10 is a diagram showing an example of a data configuration of coefficient information in Embodiment 2.

FIG. 11 is a flowchart showing pointer movement control processing in Embodiment 2.

### DESCRIPTION OF THE PREFERRED EMBODIMENTS

Hereinafter, suitable embodiments of the present invention will be described with reference to the drawings.

### Embodiment 1

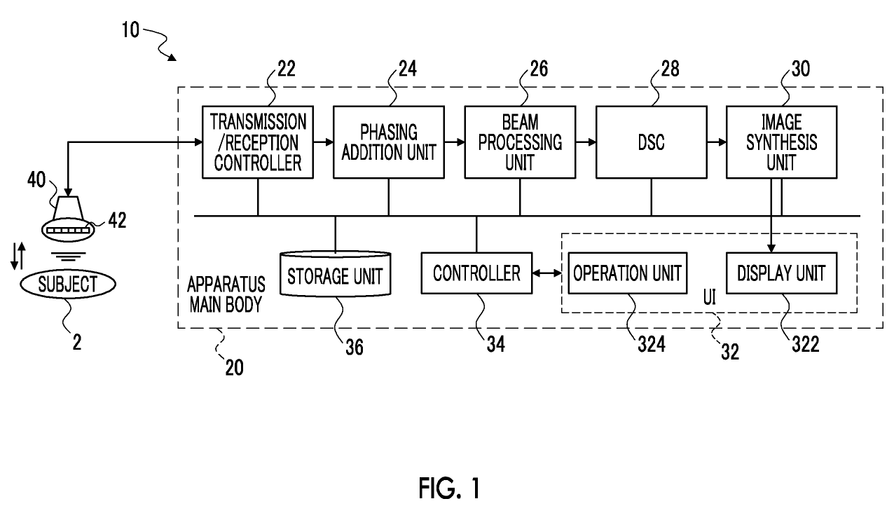

FIG. 1 is a block diagram showing a schematic configuration of an ultrasound diagnostic apparatus 10 according to the present embodiment. The ultrasound diagnostic apparatus 10 according to the present embodiment includes an apparatus main body 20 and a probe 40. The ultrasound diagnostic apparatus 10 can also be referred to as an ultrasound diagnostic system. The ultrasound diagnostic apparatus 10 has a function of executing an ultrasound diagnosis of a subject 2 using the probe 40. The apparatus main body 20 is also called a "console". The probe 40 is a device that transmits and receives an ultrasound beam for the ultrasound diagnosis. A vibration element array 42 configured by arranging a plurality of vibration elements is built in the probe 40. Each vibration element performs mutual conversion between an electric signal and an ultrasound signal by a piezoelectric effect.

The apparatus main body 20 of the ultrasound diagnostic apparatus 10 according to the present embodiment includes a transmission/reception controller 22, a phasing addition unit 24, a beam processing unit 26, a digital scan converter (DSC) 28, an image synthesis unit 30, a user interface (UI) 32, a controller 34, and a storage unit 36.

The transmission/reception controller 22 controls the transmission and reception of the ultrasound waves by each vibration element in the probe 40. The control includes, for example, supply of an electric transmission signal to each vibration element and amplification of an electric reception signal from each vibration element. In the supply of the transmission signal, the transmission/reception controller 22 controls a supply timing of the transmission signal to each vibration element to form a transmission beam of the ultrasound wave.

The phasing addition unit 24 performs phasing addition processing on the reception signal from each vibration element in the probe 40. The phasing addition processing forms a reception beam. The phasing addition unit 24 outputs echo data obtained along the reception beam as a result of the phasing addition processing.

The beam processing unit 26 performs various types of signal processing, such as gain correction processing, logarithmic amplification processing, envelope detection processing, and filter processing, on the echo data output by the phasing addition unit 24. As a result, beam data corresponding to each echo data is formed.

The digital scan converter (DSC) 28 has a coordinate transformation function and an interpolation function, and forms a display frame, that is, an ultrasound image based on a plurality of pieces of beam data output from the beam processing unit 26. The beam data from the beam processing unit 26 is data in a coordinate system of the beam scanning (hereinafter, referred to as a "scanning coordinate system") and is configured by a plurality of data points along a direction of a beam corresponding to the beam data. The DSC 28 plots, for example, a signal value of each data point of the beam data at a position of the data point in a display coordinate system, that is, a coordinate system of the ultrasound image (a rectangular coordinate system represented by a set of an x coordinate and a y coordinate in general). The plotting is performed by data writing to an internal image memory that holds a signal value of each pixel according to the display coordinate system. Then, the DSC 28 interpolates a value of a pixel having no value in the image memory after the beam data is written, from values of surrounding pixels. Such a coordinate transformation and interpolation form an ultrasound image such as a B-mode tomographic image.

The image synthesis unit 30 synthesizes an image or a character indicating various types of information with the ultrasound image formed by the DSC 28 to form display screen data. Examples of the information to be synthesized with the ultrasound image include an ROI representing a display range of various display modes such as a color Doppler mode, a sample volume of a pulse Doppler mode, a line indicating a beam on which the sample volume is positioned, and the like.

The user interface 32 corresponds to a console panel and includes a display unit 322 and an operation unit 324. The display unit 322 is a device that performs image display and is configured by, for example, a liquid crystal panel or an organic EL panel. The display screen data formed by the image synthesis unit 30 is displayed on the display unit 322. The operation unit 324 is a device operated by an operator, such as a surgeon (hereinafter, also referred to as a "user"), to input information related to a parameter or the like in the ultrasound diagnosis or a subject and to control display. The operation unit 324 is configured by various buttons, a trackball, and the like. The trackball is an example of a pointing device. In a case in which the trackball is operated by the user, the pointer displayed on the display unit 322 moves in the operation direction.

The controller 34 controls the execution of the ultrasound diagnosis processing by controlling an operation of each component included in the apparatus main body 20. In the present embodiment, the controller 34 performs the movement control of the pointer. The storage unit 36 stores screen information or configuration information described below. Details of various types of information used in the present embodiment will be described below.

The functions provided by the components 22 to 34 in the apparatus main body 20 are implemented by a cooperative operation of a computer mounted on the apparatus main body 20 and a program operating on a processor mounted on the computer. The computer may be configured to be equipped with a plurality of processors and to appropriately delegate and execute the functions provided by the components 22 to 34 to the processors. The plurality of processors may include a processor specialized in a specific processing function. The controller 34 may be implemented by a single processor or a plurality of processors. The storage unit 36 is implemented by a storage device such as a hard disk drive (HDD) mounted on the apparatus main body 20. Alternatively, the storage unit 36 may be implemented by using an external storage device via a network.

The hardware configuration of the ultrasound diagnostic apparatus 10 described with reference to FIG. 1 may basically be the same hardware configuration as before.

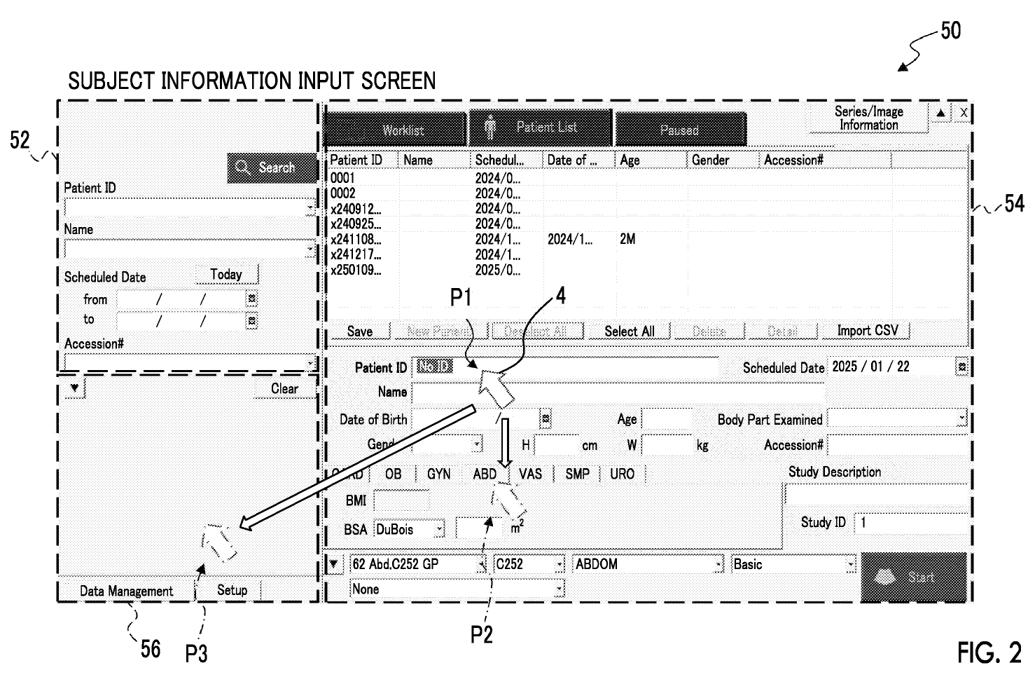

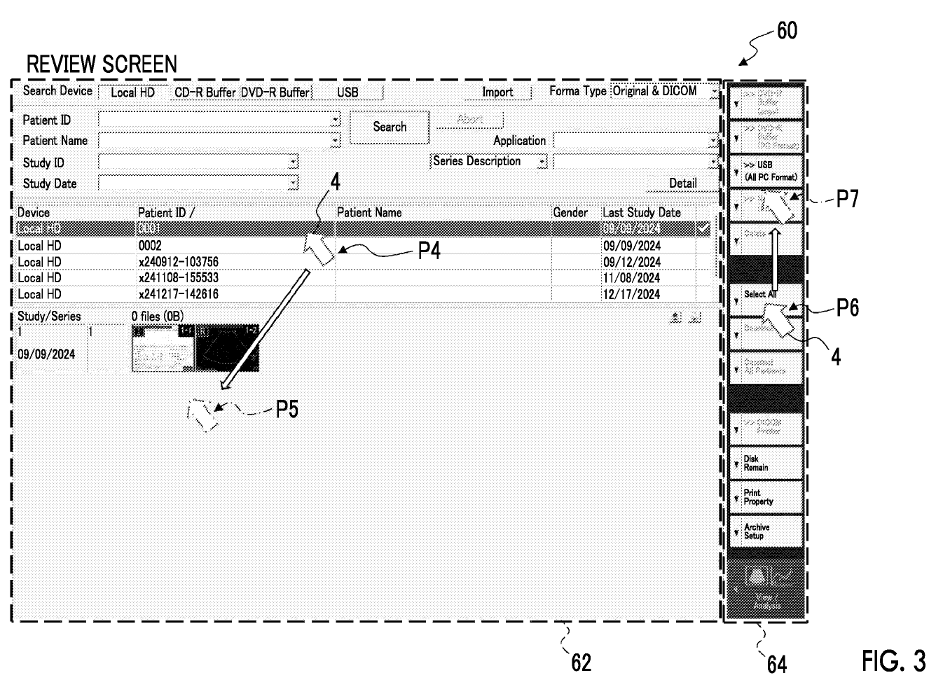

Before describing the operation in the present embodiment, a concept of the control of the movement speed according to the position of the pointer in the present embodiment will be described with reference to FIGS. 2 and 3. In the present embodiment, the trackball will be described as an example of the pointing device.

FIG. 2 is a diagram showing an example of a subject information input screen 50. The subject information input screen 50 is displayed on the display unit 322 in response to the user operation. In addition, the pointer 4 is superimposed and displayed on the subject information input screen 50 on the display unit 322.

The subject information input screen 50 is configured by being divided into three display regions 52, 54, and 56. A search screen is displayed in the display region 52. The display region 54 includes a region positioned in an upper row and displaying information related to the subject that is the examination result (hereinafter, also referred to as "subject information") in a list, and a region positioned in a lower row and serving as an item input screen for registering the subject information. A button is displayed in the display region 56.

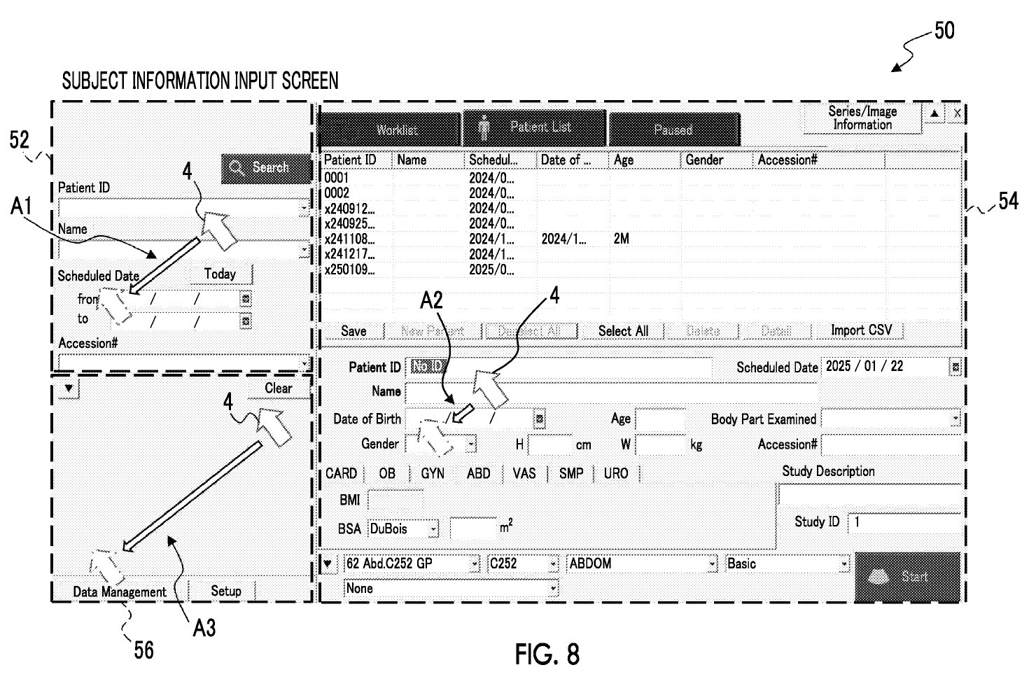

As shown in FIG. 2, it is assumed that the pointer 4 is at a position P1 in the display region 54. Since the user has input the "Patient ID", it is assumed that the user wants to move the pointer 4 to an input field of "Name" positioned directly below. In this case, the user operates the trackball to move the pointer 4 downward, but in this case, the pointer 4 may move beyond the expected amount and pass through the position of the desired input field of "Name" to move to a position P2, that is, may move too far. In FIG. 2, a pointer with a one-dot chain line indicates the position of the pointer after the movement. The same applies to FIGS. 3 and 8 described below. On the other hand, since the user has input the "Patient ID", it is assumed that the user wants to move the pointer 4 to a button of "Data Management" in the display region 56. In this case, the user operates the trackball to move the pointer 4 in a left-downward direction, but in this case, the pointer 4 may not move beyond a position P3 even though the trackball is rotated vigorously. In this case, the user needs to further rotate the trackball in the same direction.

FIG. 3 is a diagram showing an example of a review screen 60. The review screen 60 is a screen used to check the ultrasound image or the like obtained by imaging, and is displayed on the display unit 322 in response to the user operation. In addition, the pointer 4 of the pointing device is superimposed and displayed on the review screen 60 on the display unit 322. In FIG. 3, for convenience of description, two pointers 4 are shown, but only one pointer 4 is displayed at the same time. The review screen 60 is configured by being divided into two display regions 62 and 64. The display region 62 includes a region positioned in an upper row and displaying the search screen, a region positioned in a middle row and displaying a search result, and a region displaying information such as an ultrasound image of the subject selected from a list of the subject information that is the search result. Buttons are displayed in a single column in the display region 64.

As shown in FIG. 3, it is assumed that the pointer 4 is at a position P4 in the display region 62. Since the user has selected the subject from the list of the subject information, it is assumed that the user wants to move the pointer 4 to a position for selecting an image to be reviewed, which is positioned in a lower row. In this case, the user operates the trackball to move the pointer 4 downward, but in this case, the pointer 4 may move beyond the expected amount and pass through the display position of the desired image to move to a position P5, that is, may move too far. In addition, it is assumed that the pointer 4 is at a position P6 in the display region 64. After the user selects the "Select All" button, it is assumed that the user wants to move the pointer 4 to a button of "DVD-R Buffer (Copy)”. In this case, the user operates the trackball to move the pointer 4 upward, but in this case, the pointer 4 may not move beyond a position P7 even though the trackball is rotated vigorously. In this case, the user needs to further rotate the trackball in the same direction.

As described above, in a case in which the movement speed of the pointer 4 with respect to the operation of the trackball is constant, the pointer 4 may not be able to be aligned with the desired position. Therefore, in the present embodiment, the movement speed of the pointer 4 can be controlled according to the position of the pointer 4. In the present embodiment, the movement speed is controlled for each display region constituting the screen.

Incidentally, in a case in which the user performs the same operation on the trackball, that is, rotates the trackball by the same amount within the same period, it can be said that the movement speed of the pointer 4 is relatively fast in a case in which the movement amount (which can also be referred to as a "movement distance") of the pointer 4 is relatively large. On the other hand, it can be said that the movement speed of the pointer 4 is relatively slow in a case in which the movement amount of the pointer 4 is relatively small.

Subsequently, information used to perform the movement control of the characteristic pointer 4 in the present embodiment will be described. The information described below is stored in the storage unit 36.

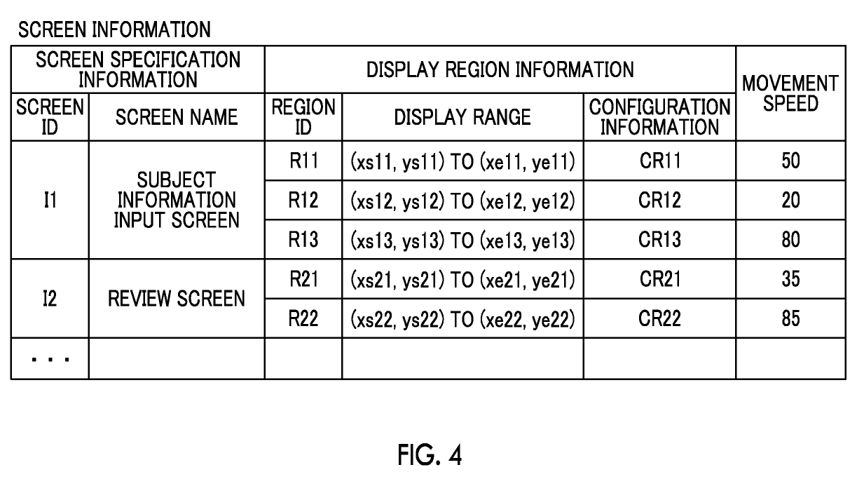

FIG. 4 is a diagram showing an example of a data configuration of screen information in the present embodiment. The screen information is information generated for each screen. The "screen" here strictly refers to software-forming content displayed on the display unit 322, and does not refer to a physical display device such as a liquid crystal panel.

The screen information is generated by associating screen specification information with display region information and the movement speed. In FIG. 4, information items not used for the description of the present embodiment are omitted. The screen specification information is information for specifying the displayed screen, and includes a screen ID for identifying the screen and a screen name. The display region information is set to information related to the display region included in the screen. The screen in the present embodiment is configured by one or a plurality of display regions. The display region information includes a region ID for identifying the display region, a display range of the display region on the screen, and configuration information. Assuming a case in which the screen and the display region are set in a rectangular shape, in a case in which the position on the screen is represented by two-dimensional coordinates with a reference point of the screen, for example, an upper left corner as an origin, the display range of the display region can be represented by coordinates of an upper left corner and coordinates of a lower right corner of the display region. The configuration information is set to information related to the content included in the display region. The movement speed is set to a movement speed in a case in which the pointer is positioned in the display region. In the present embodiment, the movement speed of the pointer 4 is manually set for each display region constituting the screen by the user.

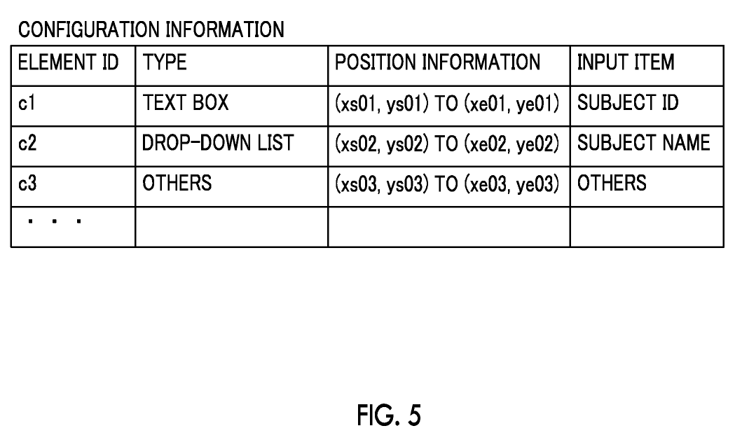

FIG. 5 is a diagram showing an example of a data configuration of the configuration information in the present embodiment. The configuration information is information set in association with each display region in the screen information as shown in FIG. 4. The configuration information is information related to a display element constituting the display region. The configuration information is generated by associating a type, position information, and an input item as an attribute of the display element with an element ID for identifying the display element. The type is information indicating a type of the display element. The position information is information for specifying a display position of the display element. As in the display range of the display region in the screen information, a reference point may be an upper left of the screen or a reference point may be an upper left of the display region. The position information of the display element can be represented in a range of coordinates of an upper left corner and coordinates of a lower right corner of the display region in a case in which the reference point is set as the origin. The input item indicates a type of the information item to be input in a case in which the display element requires input of some information.

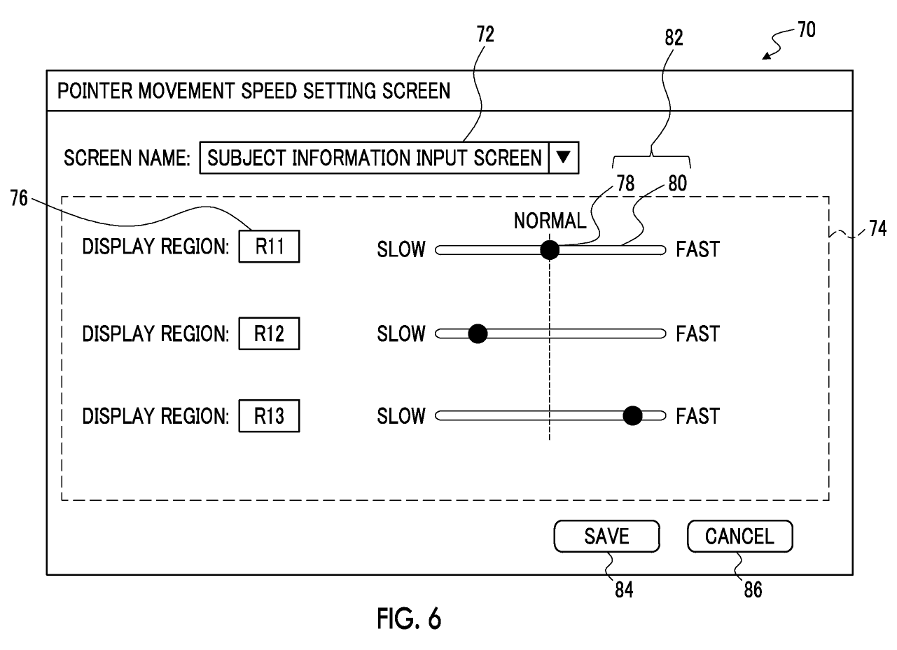

FIG. 6 is a diagram showing an example of a layout configuration of a pointer movement speed setting screen 70 in the present embodiment. The user displays the pointer movement speed setting screen 70 on the display unit 322 shown in FIG. 1 and sets the movement speed for each display region of the screen. Of course, in a case in which an initial value is set in advance for the movement speed and the movement speed set for the display region may be the initial value, the movement speed does not need to be set for the display region from the pointer movement speed setting screen 70.

The pointer movement speed setting screen 70 includes a screen selection region 72 and a movement speed setting region 74. The screen selection region 72 is formed by a pull-down menu, and a screen name of the screen registered in the screen information is displayed in a selectable manner. In the movement speed setting region 74, a display field 76 of a region ID of the display region included in the screen selected in the screen selection region 72, and a speed setting unit 82 consisting of a slider 78 and a slider bar 80 for setting the movement speed to the display region are displayed for each display region.

In a case in which the user selects the screen name to be set in the screen selection region 72, the controller 34 displays the display field 76 and the speed setting unit 82 associated with the display field 76 in the movement speed setting region 74 as many times as the number of display regions included in the selected screen, with reference to the screen information of the selected screen. Then, the movement speed set for each display region is read out from the screen information, and the slider 78 is displayed at a position of the slider bar 80 corresponding to the read-out movement speed. In a case of the first setting, the slider 78 may be displayed at the initial value. Then, the user slides the slider 78 to a position of the desired movement speed on the slider bar 80 to set the movement speed to the display region.

In a case of storing the content set by the user, the user selects a save button 84. As a result, the movement speed included in the screen information is updated by the movement speed set on the pointer movement speed setting screen 70. In a case in which the set content is not to be stored, the user selects a cancel button 86.

In the present embodiment, the speed setting unit 82 is formed by the slider 78 and the slider bar 80, and the movement speed can be set steplessly, but the user interface is not limited thereto. For example, the movement speed may be set to a numerical value within a range of upper and lower limits (for example, 0 to 100). Alternatively, the movement speed may be selected in two stages of "fast" and "slow" by a radio button, or may be set in three stages of "fast", "normal", and "slow" or in more stages.

In addition, in the present embodiment, the movement speed can be set in a batch for the display region included in one screen from the pointer movement speed setting screen 70, but the user interface for setting the movement speed is not limited thereto. For example, in a case in which the screen shown in FIGS. 2 or 3 is displayed, the movement speed may be set or reset to the display region in which the movement speed is to be set or reset by moving the pointer 4 to the display region and performing a predetermined selection operation such as a right click.

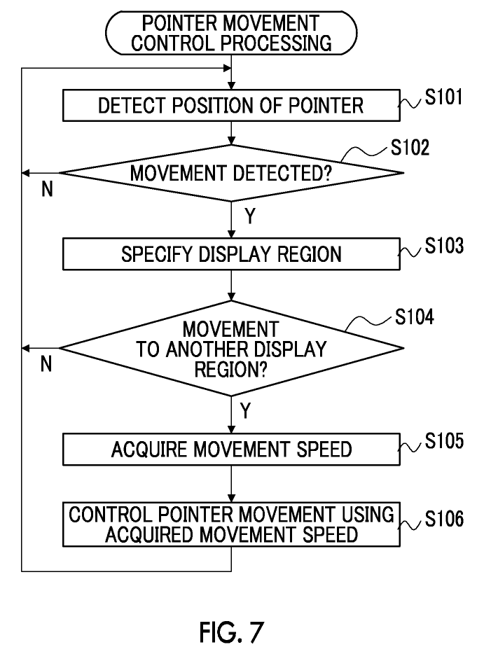

Next, processing of realizing the movement control of the pointer 4 in the present embodiment will be described with reference to a flowchart shown in FIG. 7. This processing is executed by the controller 34 or in cooperation with other components under the control of the controller 34.

The controller 34 always detects the position of the pointer 4 on the screen (step S101). In this case, the controller 34 performs processing of obtaining the coordinate position of the pointer 4 on the screen. In a case in which the user operates the trackball to move the pointer 4, the controller 34 detects the movement of the pointer 4 (Y in step S102).

The movement speed of the pointer 4 in a case in which the movement is detected is the movement speed set in the display region corresponding to the current position detected in step S101, and is set with reference to the screen information. The controller 34 can detect the movement of the pointer 4 by temporarily storing the coordinate position of the pointer 4 and comparing the detected coordinate position with the coordinate position detected immediately before. As described above, the controller 34 in the present embodiment has a function as a pointer position detection unit. In a case in which the movement of the pointer 4 is not detected (N in step S102), the processing returns to step S101.

In a case in which the movement of the pointer 4 is detected, the controller 34 specifies which display region the pointer 4 is positioned in, with reference to the display range of each display region included in the screen information (step S103). In a case in which the movement from the display region positioned immediately before to another display region is not detected (N in step S104), that is, in a case in which the movement between the display regions is not performed, the movement speed does not need to be changed, and thus the processing returns to step S101. The presence or absence of the movement between the display regions can be determined by comparing the display region specified in step S103 and the display region specified in step S103 performed immediately before.

In a case in which the movement between the display regions is detected (Y in step S104), the controller 34 acquires the movement speed set in the specified display region with reference to the screen information (step S105), and controls the pointer 4 to move at the acquired movement speed from then on (step S106). As described above, the controller 34 moves the pointer 4 in accordance with the movement speed set in the display region by the user from the predetermined pointer movement speed setting screen 70.

FIG. 8 is a diagram showing an example of the same subject information input screen 50 as in FIG. 2. FIG. 2 shows the movement of the pointer 4 before applying the characteristic movement speed control in the present embodiment, but FIG. 8 shows the movement of the pointer 4 in a case in which the characteristic movement speed control is applied in the present embodiment. In FIG. 8, it is assumed that the display regions of the region IDs R11, R12, and R13 shown in FIG. 4 correspond to the display regions 52, 54, and 56 shown in FIG. 8, respectively. In addition, as shown in FIG. 6, it is assumed that a normal movement speed, a movement speed slower than normal, and a movement speed faster than normal are set for each of the regions R11, R12, and R13.

In FIG. 8, the movement speed of the pointer 4 set by the lengths of the arrows A1, A2, and A3 is schematically shown. Even in a case in which the user performs exactly the same operation on the trackball in each of the display regions 52, 54, and 56, the movement amount of the pointer 4, that is, the movement speed indicated by the lengths of the arrows A1, A2, and A3 is different. That is, since the movement speed in the display region 54 is slow with respect to the movement speed of the pointer 4 in the display region 52, the movement amount is small. On the other hand, since the movement speed in the display region 56 is fast, the movement amount is large.

As described above, according to the present embodiment, the pointer 4 can be moved at the movement speed set in the display region including the current position of the pointer 4. As a result, even in a case in which the user performs the same operation on the trackball, the pointer 4 moves fast or slow depending on the position of the pointer 4. In a case in which the pointer 4 moves across a plurality of display regions having different movement speeds set for the pointer 4 while the user continuously rotates the trackball in the same direction, the pointer 4 moves fast or slow depending on the position of the pointer 4. That is, the pointer 4 moves while changing the movement speed.

Although the number of items of information displayed on the screen (for example, the number of subjects, the number of ultrasound images, and the like) may change, the layout of each screen is basically determined as described with reference to FIGS. 2 and 3. Specifically, each screen is formed of which display region, and each display region includes, for example, a display element such as an input field, a display field, and a selection button, and the display position of the display element is also determined in advance.

In a case in which the user has a prior understanding of the configuration of the display region in each screen, the display element constituting each display region, and the features of each screen, the user can set the desired movement speed to each display region of each screen from the pointer movement speed setting screen 70 shown in FIG. 6. Of course, since the user manually sets the desired movement speed in each display region, any movement speed can be set without considering the characteristics of the display region or the like.

The information shown in FIGS. 4 and 5 may be displayed on the display unit 322 so that the user can refer to the information in a case in which the movement speed is set.

Incidentally, even in the related art, in a case in which the user operates the trackball to rotate the trackball quickly, the pointer may also move quickly. However, this is equivalent to an operation of increasing the rotation amount by rotating the trackball quickly, and the pointer simply moves faster and farther by increasing the rotation amount.

Changing the "movement speed" is equivalent to changing the movement amount of the pointer with respect to the operation amount of the pointing device. Since the speed is set for the movement of the mouse cursor of the PC, the movement speed is set instead of the movement amount in the present embodiment.

In addition, in a case in which the movement speed is set for each display region, the movement speed is not limited to a method of setting the movement speed in association with the display region. For example, a "region attribute" is provided as a type of the region. Then, the movement speed is set for each region attribute. For example, three types of attributes of a normal region (Standard Zone), a high-precision region (High Precision Zone), and a rapid operation region (Rapid Operation Zone) are prepared as the region attributes. The normal region is a region in which a normal operation can be performed, and the pointer movement speed is set to a normal setting, that is, is set to be equal to the initial setting of the system. The high-precision region is a region in which an accurate operation is required, and thus the pointer movement speed is set to be slower than that in the normal region. The rapid operation region is a region in which a rapid operation can be performed, and thus the pointer movement speed is set to be faster than that in the normal region.

Then, the user sets one of the three region attributes for each display region. This setting may be substantially the same as setting any of the movement speeds of the three stages of "fast", "normal", and "slow" described above. However, it is also considered that, for the user who understands the configuration of the screen, it is easier to set the region attribute for the display region than to directly set the movement speed. In this case, in the screen information shown in FIG. 4, the "region attribute" is set instead of the "movement speed".

As described above, in the present embodiment, the movement speed is set for each display region constituting the screen. However, the movement speed is not limited to being set for each display region. For example, one display region may be finely divided into a plurality of regions, and the movement speed may be set for each finely divided region. Even in one display region, for example, there may be a case in which a region in which it is not desired to move the pointer 4 largely because the input field is densely provided and a region in which the input field is not provided so much may be mixed. This is because the screen is not necessarily set in consideration of the movement of the pointer 4 based on the user operation. The user sets the display region to be divided into a plurality of small regions, and sets the movement speed for each small region.

In addition, the user may divide the screen into any small regions regardless of the display region constituting the screen, and set the movement speed for each divided small region.

In the present embodiment, since the movement speed of the pointer 4 is controlled with reference to the display region according to the layout of the screen, the user does not need to separately set the region as the unit of the movement speed setting. However, as in the modification example described above, the movement speed of the pointer 4 may be controlled for each any region by setting the region as the unit of the movement speed control for the user.

### Embodiment 2.

In Embodiment 1, the user sets the movement speed to each display region from the pointer movement speed setting screen 70 shown in FIG. 6. In the present embodiment, the movement speed of the pointer is controlled according to the position of the pointer on the screen, and the movement speed is automatically set. The configuration of the ultrasound diagnostic apparatus 10 in the present embodiment may be the same as that in Embodiment 1. In the present embodiment, the content of the control in the controller 34 and the information used are slightly different from those in Embodiment 1.

As a method of automatically setting the movement speed, in the present embodiment, the movement speed of the pointer is controlled based on the type of the display element present around the position of the pointer. Specifically, the processing is performed as follows.

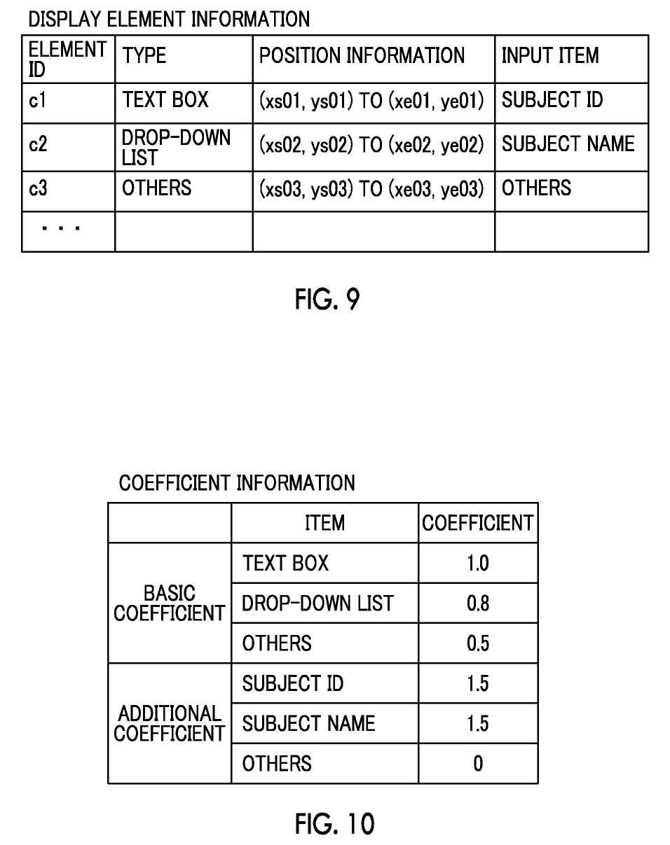

FIG. 9 is a diagram showing an example of a data configuration of display element information referred to in a case of performing the movement control of the pointer in the present embodiment. The display element information is information related to an element displayed on the screen, and is, for example, information related to an input field or a selection button of the information, a region in which the information is displayed in a list, and the like. In the subject information input screen 50 shown in FIG. 2, an input field of each item information of the subject information such as a search keyword and "Name", a display field in which the information items such as the subject information are displayed in a list in a selectable manner by the user, a selection button selected by the user such as a "Data Management" button, and the like correspond to the display elements.

The display element information is generated by associating a type, position information, and an input item as an attribute of the display element with an element ID for identifying the display element. Since the type, the position information, and each information item of the input item are the same as the configuration information shown in FIG. 5, the description thereof will be omitted. The display element information is basically the same information as the configuration information used in Embodiment 1, but does not need to be associated with the display region.

FIG. 10 is a diagram showing an example of a data configuration of coefficient information used in the present embodiment. In the coefficient information, a coefficient that is referred to in a case of calculating an evaluation value for evaluating what kind of position the current position of the pointer 4 is, that is, a position attribute of the pointer 4 is set. The coefficient information is classified into a basic coefficient and an additional coefficient. The basic coefficient is a coefficient set in association with an item value set for the type included in the display element information. The additional coefficient is a coefficient set in association with an item value set for the input item included in the display element information. Both the basic coefficient and the additional coefficient are set in advance by the user. In the present embodiment, the higher the evaluation value, the higher the evaluation, but the opposite may also be used.

In the present embodiment, the movement speed to be set as the movement speed of the pointer 4 needs to be set in advance. For example, as described in Embodiment 1, three types of region attributes of a normal region in which a normal movement speed is adopted, a high-precision region in which a movement speed slower than the normal region is adopted, and a rapid operation region in which a movement speed faster than the normal region is adopted are prepared as the region attributes. Of course, the three types of movement speeds are not limited, and a plurality of movement speeds may be prepared. In the present embodiment, the movement speed is set by determining which region attribute the current position of the pointer 4 corresponds to.

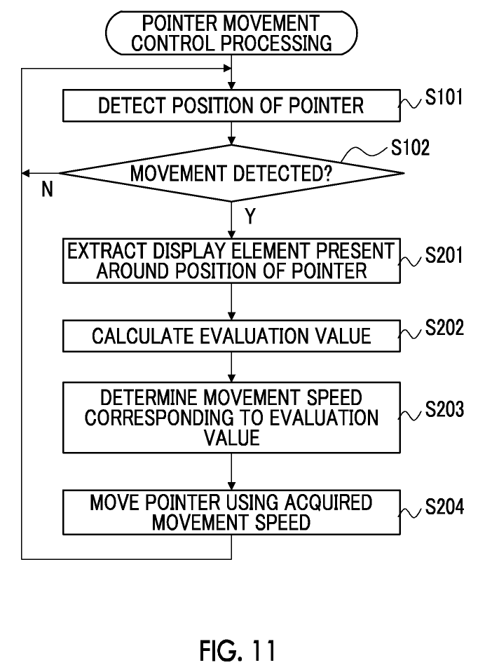

Next, processing of realizing the movement control of the pointer 4 in the present embodiment will be described with reference to a flowchart shown in FIG. 11. This processing is executed by the controller 34 or in cooperation with other components under the control of the controller 34. The same processing as in Embodiment 1 is assigned the same step number, and the description thereof will be appropriately omitted.

The controller 34 always detects the position of the pointer 4 until the movement of the pointer 4 on the screen is detected (N in steps S101 and S102). In a case in which the user operates the trackball to move the pointer 4, the controller 34 detects the movement of the pointer 4 (Y in step S102).

In a case in which the movement of the pointer 4 is detected, the controller 34 extracts the display element present around the position of the pointer 4 (step S201). Therefore, the controller 34 collates the current position coordinate of the pointer 4 with the position information of each display element set in the display element information. Since the display element has a range as indicated by the position information, the controller 34 may collate the current position coordinate of the pointer 4 with, for example, the coordinate of the center point of the display element. Then, the controller 34 sets a predetermined range centered on the current position of the pointer 4, for example, within 10 pixels of the display unit 322, as the position around the pointer 4, and extracts the display element present around the position of the pointer 4.

Subsequently, the controller 34 evaluates the position attribute of the pointer 4, that is, what kind of position the current position of the pointer 4 is. The evaluation value that objectively shows the evaluation of the current position of the pointer 4 is calculated as follows.

First, the controller 34 extracts the type of each extracted display element with reference to the display element information, and calculates the evaluation value with reference to the extracted type and the coefficient information shown in FIG. 10 (step S202).

In a case of calculating the evaluation value, first, the evaluation value related to the basic coefficient is calculated. Specifically, the number of each type of the display element and the coefficient set for the type are multiplied. For example, in a case in which there are five display elements of the type of the text box around the current position of the pointer 4, the evaluation value for the text box can be calculated as 5 × 1.0 = 5. The same calculation is performed for other types.

In addition, the evaluation value related to the additional coefficient is calculated. Specifically, the number of each input item of the display element and the coefficient set for the input item are multiplied. For example, in a case in which there are four display elements of the input item of the subject ID around the current position of the pointer 4, the evaluation value for the subject ID can be calculated as 4 × 1.5 = 6. The same calculation is performed for other input items.

The sum of the results calculated as described above is treated as the evaluation value. Then, the controller 34 compares the calculated evaluation value with a predetermined threshold value to determine the movement speed of the pointer 4.

In the present embodiment, since any of the normal movement speed adopted in the normal region, the movement speed slower than the normal region adopted in the high-precision region, or the movement speed faster than the normal region adopted in the rapid operation region is set as the movement speed of the pointer 4, two threshold values are set in advance as the predetermined threshold values. Then, the movement speed to be set in the pointer 4 is determined in accordance with the result of comparing the evaluation value with the predetermined threshold value (step S203). For example, in a case in which the evaluation value is equal to or larger than the first threshold value (for example, 2.5), the movement speed is determined to be the slow movement speed adopted in the high-precision region. In a case in which the evaluation value is smaller than the first threshold value (for example, 2.5) and larger than the second threshold value (for example, 0.5), the movement speed is determined to be the normal movement speed adopted in the normal region. In addition, in a case in which the evaluation value is equal to or smaller than the second threshold value, the movement speed is determined to be the fast movement speed adopted in the rapid operation region. In the present embodiment, the higher the evaluation, the slower the movement speed of the pointer 4 is set.

For example, since the input field is dense in the position of the pointer 4 in the display region 54 shown in FIG. 8, the slow movement speed is set for the pointer 4 at this position. In addition, since the input field is not dense in the position of the pointer 4 in the display region 56 shown in FIG. 8, the fast movement speed is set for the pointer 4 at this position.

In a case in which the movement speed to be set for the movement of the pointer 4 is determined as described above, the controller 34 moves the pointer 4 at the movement speed (step S204).

The fact that the evaluation value is large means that a display element such as a text box or a drop-down list, for which the user needs to align the pointer 4 at a desired position to input information or to select an information item, is present around the position of the pointer 4 or is relatively large in number. Therefore, the movement speed of the pointer 4 is relatively slow. On the contrary, it means that a display element for which the user needs to input or select information is not present around the position of the pointer 4 or is relatively small in number. Therefore, the movement speed of the pointer 4 is relatively fast with priority to the alignment of the pointer 4 over the moving distance.

According to the present embodiment, the movement speed can be set according to the attribute of the display element present around the current position of the pointer 4 and the number of each attribute.

The control of the movement speed of the pointer 4 described in each of the above-described embodiments has been described in a case of being applied to the user interface 32 in the ultrasound diagnostic apparatus 10, but can also be applied to other medical apparatuses having a pointing device. In addition, the present invention is not limited to the medical apparatus, and may be applied to a general computer such as a PC having a mouse or a touchpad as a pointing device.

In the present embodiment, each processing is executed by any computer. In addition, any computer may execute these processes using a processor as hardware, a program as software, or a combination thereof. In such a case, the processor is configured to execute various types of processing in the present embodiment in cooperation with the program, and may function as each unit or each means in the present embodiment. Additionally, the execution order of the process by the processor is not limited to the order described above and may be changed as appropriate. Any computer may be a general purpose computer, a special purpose computer, a workstation, or other system capable of executing each process.

The processor may be configured with one or more pieces of hardware, and the type of hardware is not limited. For example, the processor can be configured by hardware such as a central processing unit (CPU), a micro processing unit (MPU), a programmable logic device such as a field-programmable gate array (FPGA), a dedicated circuit for executing specific processing such as an application-specific integrated circuit (ASIC), a graphics processing unit (GPU), or a neural processing unit (NPU). In addition, the types of hardware may be a combination of different types of hardware. In a case where a plurality of hardware components are configured to execute one or a plurality of processes of a certain processor, the plurality of hardware components may be present in physically separate devices or may be present within the same device. In addition, in any of the embodiments, the order of the processes performed by the processor is not limited to the order described above, and may be changed as appropriate. In addition, hardware is implemented in a form of an electric circuit (circuitry) in which circuit elements, such as semiconductor elements, are combined.

Further, the program may be software, such as firmware or a microcode. The program may also be, for example, a group of program modules, and each function may be implemented by a processor configured to execute the corresponding function. The program may be a program code or a plurality of code segments that are stored in one or a plurality of non-transitory computer-readable media (for example, storage media or other storages). The program may be divided and stored in a plurality of non-transitory computer-readable media present in devices physically separated from each other. The program code or the code segments may represent any combination of a procedure, a function, a subprogram, a routine, a subroutine, a module, a software package, a class, an instruction, a data structure, and a program statement. The program code or code segments may be connected to other code segments or hardware circuits by transmitting and receiving information, data, arguments, parameters, or contents of a memory. In addition, the present invention can also be applied to a program and a program product.

What is claimed is:

1. An ultrasound diagnostic apparatus comprising a processor,
wherein the processor is configured to control a movement speed of a pointer that moves on a screen on which information related to an ultrasound diagnosis is displayed in accordance with a display position of the pointer, the pointer being moved by operation of a pointing device.

2. The ultrasound diagnostic apparatus according to claim 1,
wherein the movement speed of the pointer is set for each region constituting the screen.

3. The ultrasound diagnostic apparatus according to claim 2,
wherein the processor moves the pointer in accordance with the movement speed set for each region by a user via a predetermined setting screen.

4. The ultrasound diagnostic apparatus according to claim 1,
wherein the processor controls the movement speed of the pointer based on an attribute of a display element present around a position of the pointer.

5. The ultrasound diagnostic apparatus according to claim 4,
wherein the processor controls the movement speed of the pointer in accordance with a result of comparing a position attribute of the pointer obtained from the attribute of the display element with a predetermined threshold value.
